SEQUENCE LISTING (21 sequences)

SEQ ID NO: 1 - protein, 28 aa, Homo sapiens
  misc_feature (2)..(2): Xaa can be any naturally occurring amino acid
  misc_feature (27)..(28): Xaa can be any naturally occurring amino acid
MXDSHAKRHHGYKRKFHEKHHSHRGYXX

SEQ ID NO: 2 - protein, 28 aa, Homo sapiens
MGDSHAKRHHGYKRKFHEKHHSHRGYDV

SEQ ID NO: 3 - protein, 24 aa, Homo sapiens
  misc_feature (1)..(2): Xaa can be any naturally occurring amino acid
  misc_feature (23)..(24): Xaa can be any naturally occurring amino acid
XXLAHQKPFIRKSYKCLHKRCRXX

SEQ ID NO: 4 - protein, 22 aa, Homo sapiens
GCLAHQKPFIRKSYKCLHKRCR

SEQ ID NO: 5 - protein, 41 aa, Homo sapiens
GIGDPVTCLKSGAICHPVFCPRRYKQIGTCGLPGTKCCKKP

SEQ ID NO: 6 - protein, 134 aa, Camelus dromedarius
DVQLQESGGASVQPGGSLLISCEASGLASYSNYCIMWFRQPPGKEREGVAGINLRSGITYYAEAVRPRFTISADSVDGRFAISQDNARNTVYLQMNSLKPEDTAIYYCAAGNLCGGSWSGYRYWGQGTQVTVSS

SEQ ID NO: 7 - protein, 127 aa, Camelus dromedarius
DVQLQESGGGLVQPGGSLRLSCRASGWTADNWYMGWFRQSPGKEREAVAIIGHRFDTTYYADSVQGRFTITQDNVEKMVFLEMNNLKPEDTAMYYCAIQVYNGGVRPSPDAAKYNYWGQGTQVTVSS

SEQ ID NO: 8 - protein, 126 aa, Camelus dromedarius
DVQLQESGGASVQVGGSLTLSCSTSKVPNIGCVTWFRQGPGGLQVGIAAVRTRYGDTYYQDSIKGRFTISRTHTTENLQMNALEPDDAAVYRCATTSKSSCYSGGSWTLEDVYEYWGQGTQVTVSS

SEQ ID NO: 9 - protein, 127 aa, Camelus dromedarius
DVQLQDSGGESVQAGGSLRLTCVGSGNSFIRYCMAWFRQAPGKQREEIVESGQFEFQTWNPDSVKGRFTISRDNAPNTGSLHMNSLQSEDTAAYFCAAGMICPIFGRTQMSADMDYWGRGTQVTVSS

SEQ ID NO: 10 - protein, 128 aa, Camelus dromedarius
SVQPGGSLLISCEASGLASYSNYCIMWFRQPPGKEREGVAGINLRSSITYYADSVKARFTISADSVEGRFAISQDKSRNTVLLQMNSLKAEDTANYYCAAAVCQSRYMAHDQVSYNYWGQGTQVTVSS

SEQ ID NO: 11 - protein, 120 aa, Camelus dromedarius
DVQLQESGGGSVQAGGSLRLSCVVSEYRACMGWFRQAPGKEREAVAVIGSGTSTYTADSVKGRFTISRDNDRKTATLQMDSLEPEDTAIYYCAVGRNCKWPPLNFGATTWGQGTQVTVSS

SEQ ID NO: 12 - protein, 6 aa, Artificial Sequence
  Elastase B cleavage seqeunce
RGGGLA

SEQ ID NO: 13 - protein, 6 aa, Artificial Sequence
  Elatase B cleavage sequence
AGGLAP

SEQ ID NO: 14 - protein, 6 aa, Artificial Sequence
  signal peptidase 3 cleavage sequence
ASAALA

SEQ ID NO: 15 - protein, 13 aa, Artificial Sequence
  Neutrophil protease cleavage sequence
NATLDPRSFLLRN

SEQ ID NO: 16 - protein, 5 aa, Artificial Sequence
  GS linker sequence
GGGGS

SEQ ID NO: 17 - protein, 15 aa, Artificial Sequence
  GS linker sequence
GGGGSGGGGSGGGGS

SEQ ID NO: 18 - protein, 9 aa, Artificial Sequence
  Linker Seqeunce
  MISC_FEATURE: Where Glu is Glu or Asp
EEGEFSEAR

SEQ ID NO: 19 - protein, 17 aa, Artificial Sequence
  Linker Seqence
GSADDAKKDAAKKDGKS

SEQ ID NO: 20 - protein, 20 aa, Artificial Sequence
  Linker Sequence
SSADDAKKDAAKKDDAKKDA

SEQ ID NO: 21 - protein, 187 aa, Artificial Sequence
  Antibody fragment based antimicrobial conjugate
MGDSHAKRHHGYKRKFHEKHHSHRGYDVRGGGLAGGGGSGGGGSGGGGSHMDVQLQESGGASVQPGGSLLISCEASGLASYSNYCIMWFRQPPGKEREGVAGINLRSGITYYAEAVRPRFTISADSVDGRFAISQDNARNTVYLQMNSLKPEDTAIYYCAAGNLCGGSWSGYRYWGQGTQVTVSSLE

(19) United States
(12) Patent Application Publication
(10) Pub. No.: US 2024/0091375 A1
(43) Pub. Date: Mar. 21, 2024

(54) AN ANTIBODY FRAGMENT BASED ANTIMICROBIAL CONJUGATE SELECTIVELY TARGETING PSEUDOMONAS

(71) Applicant: ABGENICS LIFESCIENCES PRIVATE LIMITED, Pune (IN)

(72) Inventor: SANJIBAN KUMAR BANERJEE, Pune (IN)

(73) Assignee: ABGENICS LIFESCIENCES PRIVATE LIMITED, Pune (IN)

(21) Appl. No.: 17/998,345

(22) PCT Filed: Apr. 12, 2021
(86) PCT No.: PCT/IB2021/052989

§ 371 Date: Nov. 9, 2022

(30) Foreign Application Priority Data
May 17, 2020 (IN) 202021020769

(51) Int. Cl. A61K 47/68; C07K 16/12

(52) U.S. Cl. CPC A61K 47/6849; A61K 47/6811; A61K 47/6889; C07K 16/1214; C07K 2317/22; C07K 2317/569; C07K 2319/50

(57) ABSTRACT

The present invention relates to a novel antibody fragment based antimicrobial conjugate selectively targeting Pseudomonas spp., preferably Pseudomonas aeruginosa, comprising of at least one antimicrobial peptide at one end of the conjugate preferably human Histatin-5; at least one antibody fragment at the other end of the conjugate, preferably a VHH targeting C4 decarboxylase transporter antigen of Pseudomonas aeruginosa; at least one protease cleavage sequence, preferably susceptible to cleavage by Pseudomonas aeruginosa specific virulent protease, Elastase B, and at least one flexible polypeptide linker in tandem with the protease cleavage sequence, and the protease cleavage sequence and the flexible polypeptide linker placed in between the antimicrobial peptide and antibody fragment. The antibody fragment-based antimicrobial conjugate has an in vitro MIC-99 against Pseudomonas aeruginosa of 0.5 μM, and MIC-50 less than 0.125 μM. It can be easily manipulated for generating next generation of conjugates in case of emergence of drug-resistant forms of the pathogen.

### REFERENCE TO SEQUENCE LISTING

A Sequence listing in text format is incorporated by reference into the specification. The name of the text filed containing the Sequence Listing is Seq. Pseudomonas_ST25. The test file is about 13 KB, and is being submitted electronically via EFS-WEB.

### RELATED APPLICATIONS

This application claims priority from the PCT application No. PCT/IB2021/052989 filed on 12 Apr. 2021, which claims priority to the Indian provisional patent application numbered 202021020769 titled “AN ANTIBODY FRAGMENT BASED ANTIMICROBIAL CONJUGATE SELECTIVELY TARGETING PSEUDOMONAS” filed on 17^{th} of May 2020. Both applications are incorporated in full herein by reference.

### FIELD OF THE INVENTION

The present invention relates to an antibody fragment based antimicrobial conjugate selectively targeting Pseudomonas spp.

### BACKGROUND OF THE INVENTION

Pseudomonas aeruginosa is an opportunistic pathogen known to cause nosocomial infections i.e., infections in people who are already hospitalized with another illness or condition, or people who have a weak immune system. Systemic infections in the urinary tract and lungs are common and it causes topical infections in burn injuries and diabetic ulcers. For severe infections, anti-pseudomonal drugs like carbapenems, fluoroquinolones, and aminoglycosides are used for therapy. Their use is however limited by the rapid appearance of drug resistant forms, particularly in hospital settings. Drug-resistance is mainly due to multiple intrinsic resistance mechanisms like beta-lactamase production, efflux-mediated and porin-related resistance, and target modification (Matteo et al., 2018, Drugs in context, DOI: 10.7573/dic.212527). Further, hyper production, or “derepression,” through chromosomal mutation, confers resistance to a number of anti-pseudomonal agents, such as piperacillin/tazobactam. Other mechanisms such as overexpression of efflux pumps (carbapenems), or downregulation of porin production (carbapenems and cefepime) are responsible for multidrug resistance primarily to fluoroquinolones and aminoglycosides (Breidenstein et al., 2011, Trends Microbiol., 19:419-426).

Present-day antibiotics focus on small molecules targeting a cellular or enzymatic component in a microbe. However, this approach is easily rendered ineffective by the pathogens by small mutations in the target genes because of which even though newer generations of smaller molecule antibiotics are constantly being developed, their inherent design shortcomings result in their having a limited use in the long run.

A particular class of antimicrobial molecules that has emerged as a solution to the drug resistance problem is the antimicrobial peptides (Hancock and Sahl, 2006, Nat. Biotechnol., 24:1551). To augment their effect, there have been efforts towards achieving target-specific antimicrobial therapy consisting of conjugating antibiotics to monoclonal antibodies or constructing large fusion proteins with bactericidal and bacterial recognition domains. These antibody-antibiotic conjugates enhance the therapeutic index by maximizing efficacy and minimizing off-target toxicity. Such conjugates comprise a targeting antibody covalently attached through a linker unit to a cytotoxic drug moiety.

U.S. Pat. No. 4,867,973A is one of the earliest citations related to antibody-therapeutic agent conjugate. The invention is related to antibody-therapeutic agent conjugates having a therapeutic agent covalently attached to an antibody or antibody fragment.

U.S. Pat. No. 7,569,677B2 describes a composition including a purified antibody conjugated with at least one antibiotic, the antibody having an antigen-binding portion that binds at least one antigen derived from Staphylococcus or Streptococcus. Here the conjugation is via a covalent bond.

U.S. Pat. No. 9,895,450B2 describes an antibody-antibiotic conjugate compound comprising an anti-wall teichoic acid (WTA) monoclonal antibody wherein the anti-wall teichoic acid monoclonal antibody binds specifically to Staphylococcus aureus, and covalently attached by a protease-cleavable, peptide linker (L) to an antibiotic.

WO2017083515A2 provides a very broad-spectrum antibody molecule-drug conjugate that specifically binds to core penta-saccharide region of lipopolysaccharides (LPS) targeting one or more Gram-negative bacteria including species of Enterobacteriaceae chosen from a species of Klebsiella, Enterobacter, Shigella, Escherichia, Salmonella, or Citrobacter, a species of Pseudomonas, a species of Acinetobacter, or any combination thereof.

However, such methods are yet to result in functional, effective therapeutics due to the low efficiency of chemical conjugation, instability of large proteins, and/or high cost of production.

Several modifications and improvements have been tried in this field of research. One such improvement is use of camelid antibodies instead of normal immunoglobulins. It has been demonstrated that, in Camelidae family (camels, dromedaries, llamas and alpacas), about 50% of immunoglobulins are antibodies devoid of light chain. These heavy-chain antibodies interact with the antigen by the virtue of only one single variable domain, referred to as VHH(s), VHH domain(s) or VHH antibody(ies), or nanobodies. Recombinant VHH domains (VHHs) exhibit the antigen-binding capacity of the camelid original heavy-chain antibody (Nguen et al., 2001, Adv. Immunol., 79, 261-96; Muyeldermans et al., 2001, Trends in Biochemical Sciences, 26:230-235).

US20060211088A1 describes a method for generating or cloning a nucleic acid or nucleotide sequence that encodes a heavy chain antibody or an antigen-binding fragment directed against a specific antigen by providing a sample or population of cells from a Camel immunized with said antigen, isolating from said sample or population said at least one cell that expresses or is capable of expressing a heavy chain antibody directed against said antigen, and obtaining from said at least one cell a nucleic acid or nucleotide sequence that encodes a heavy chain antibody directed against antigen or that encodes an antigen-binding fragment thereof directed against said antigen.

WO2010080819A1 describes novel targeted antimicrobial compositions comprising an antimicrobial peptide attached to a peptide targeting moiety that binds a bacterial strain or species. It briefly describes use of nanobodies (VHH fragments derived from immunized camels) as targeting moieties.

Szynol et. al. has reported immunoconjugate composed of the variable domain of a llama heavy chain antibody (VHH) against Streptococcus mutans and dhvar5, a synthetic antimicrobial peptide. To promote in vivo release of the active peptide, a factor Xa cleavage site was inserted between VHH and dhvar5. However, some data showed that there was diminished antimicrobial activity of dhvar5 by the N-terminal fusion to VHH (Szynol et. al., 2006, Chem Biol Drug Des., 67(6):425-31). The authors had suggested that there is requirement of more research on properties and design of the other recombinant molecules composed of peptides toxic to host cells.

The prior art does not provide a comprehensive solution to target microbial infections with high specificity and less toxicity to infected hosts. Due to continuous ability of pathogens to acquire drug-resistance, there is a technological requirement to develop a solution which can be easily manipulated and substituted to form next-generation drug to deal with resistant pathogens. Further, the prior art fails to provide large antimicrobial molecules which get activated at the site of action by releasing the peptide, converting the nontoxic prodrug to a toxic drug, which is not toxic to the host body. Moreover, the prior art fails to provide a simple solution for dealing with Pseudomonas spp. infection, more specifically, a solution for controlling the drug-resistance pattern in Pseudomonas spp.

### OBJECT(S) OF THE INVENTION

Accordingly, the present invention takes into account the drawbacks of the prior art and provides an invention with the main object of providing a novel antibody fragment based antimicrobial conjugate specifically targeting Pseudomonas spp., preferably Pseudomonas aeruginosa, comprising of at least one antimicrobial peptide at one end of the conjugate either at N-terminal or C-terminal of the conjugate, belonging to the group comprising either of cationic histidine-rich antimicrobial peptides, mucin family of proteins, or human defensins; at least one antibody fragment at the other end of the conjugate, preferably, a camelid heavy chain antibody variable region fragment (VHH) specific against surface antigen of Pseudomonas aeruginosa; and at least one protease cleavage sequence and at least one flexible polypeptide linker in tandem, with the protease cleavage sequence and flexible polypeptide linker placed in between the antimicrobial peptide and antibody fragment, wherein the protease cleavage sequence is susceptible to cleavage by proteases belonging to the group consisting of membrane, cell wall associated, or secreted proteases of Pseudomonas spp., or host neutrophil proteases.

Another object of the invention is to provide antibody fragment based antimicrobial conjugate selectively targeting Pseudomonas aeruginosa in a highly specific manner and also reducing off-target toxicity.

Yet another object of the invention is to provide an antibody fragment based antimicrobial conjugate selectively targeting Pseudomonas spp. which is a non-toxic prodrug and gets activated only when exposed to pathogenic Pseudomonas spp. after the release of the antimicrobial peptide only in the vicinity of the pathogen by the cleavage of protease cleavage sequence of the conjugate by membrane, cell wall associated, or secreted proteases of Pseudomonas spp., or host neutrophil proteases, thus causing less host toxicity or off-target toxicity due to the antimicrobial peptide.

Yet another object of the invention is to provide antibody fragment based antimicrobial conjugate specifically targeting Pseudomonas aeruginosa, which is effective against the drug resistant forms of P. aeruginosa, as it does not penetrate the pathogenic P. aeruginosa, and acts extracellularly by lysing or neutralizing the pathogen, a mode of action which also reduces the chance of development/mutation of pathogen to resistant forms.

Yet another object of the invention is to provide antibody fragment based antimicrobial conjugate specifically targeting P. aeruginosa, comprising of at least one antimicrobial peptide, at least one antibody fragment, preferably, a camelid VHH, at least one protease cleavage sequence, and at least one flexible polypeptide linker in tandem in either the forward or reverse order, which can be easily manipulated for generating next generation of conjugates in case of emergence of drug-resistant forms of the pathogen, wherein, the antimicrobial peptide can be changed either by mutations or can be replaced with more toxic peptides, the protease cleavage sequence can be replaced, the linker can be replaced, and the VHH can be replaced to recognize mutated pathogen more efficiently resulting in more efficient pathogen neutralization.

### SUMMARY OF THE INVENTION

In the main embodiment, the invention provides a novel antibody fragment based antimicrobial conjugate selectively targeting Pseudomonas spp., preferably Pseudomonas aeruginosa, comprising of at least one antimicrobial peptide at one end of the conjugate either at the N-terminal or C-terminal of the conjugate, belonging to the group comprising of Histatins, cationic histidine-rich antimicrobial peptides, mucin family of proteins, and human defensins; at least one antibody fragment at the other end of the conjugate, preferably, camelid VHH targeting surface antigen of Pseudomonas aeruginosa; at least one protease cleavage sequence and at least one flexible polypeptide linker in tandem, with the protease cleavage sequence and flexible polypeptide linker placed in between the antimicrobial peptide and antibody fragment, wherein the protease cleavage sequence is susceptible to cleavage by proteases selected from the group consisting of membrane, cell wall associated, or secreted proteases of Pseudomonas spp., or host neutrophil proteases. The in vitro MIC-99 (minimal inhibitory concentration to kill 99% microorganisms) of the conjugate against Pseudomonas aeruginosa is around 0.5 μM and MIC-50 is less than 0.125 μM; whereas, the MIC-99 of the VHH targeting Pseudomonas aeruginosa is around 10 μM and MIC-50 is less than 2.5 μM, making the conjugate more effective than the VHH alone against Pseudomonas aeruginosa.

The invention relates to a novel antibody fragment based antimicrobial conjugate selectively targeting Pseudomonas spp., preferably Pseudomonas aeruginosa, wherein, said conjugate acts a prodrug and gets activated only upon interaction with pathogenic Pseudomonas aeruginosa. This makes said conjugate less toxic to host cells being administered with said conjugate for treating infections with Pseudomonas aeruginosa.

The invention also relates to antibody fragment based antimicrobial conjugate selectively targeting Pseudomonas spp., preferably, Pseudomonas aeruginosa, which can be easily manipulated by replacement/substitution of components of said conjugate, wherein, the antimicrobial peptide can be changed by mutation or replaced with more toxic peptides, the protease cleavage sequence can be replaced, the linker can be replaced, and the VHH can be replaced or the order of the components can be changed which enables development of novel antibody fragment based antimicrobial conjugate which are efficient to deal with drug-resistance in Pseudomonas aeruginosa.

The antibody fragment based antimicrobial conjugates can constitute pharmaceutical compositions for topical application, systemic delivery, or oral consumption.

The antibody fragment based antimicrobial conjugates can constitute formulations for coating medical implants to reduce infections.

### BRIEF DESCRIPTION OF THE DRAWING

The object of the invention may be understood in more details and more particularly description of the invention briefly summarized above by reference to certain embodiments thereof which are illustrated in the appended drawings, which drawings form a part of this specification. It is to be noted, however, that the appended drawings illustrate preferred embodiments of the invention and are therefore not to be considered limiting of its scope, for the invention may admit to other equally effective equivalent embodiments.

FIG. 1a is a schematic of the antifungal conjugate (100) depicting the cleavage of the conjugate (100) at the protease cleavage site (103) separating antimicrobial peptide (101) and the antibody (102);

FIG. 1b is a schematic depicting the mode of action of the antifungal conjugate (100) at the surface of the pathogen cell membrane (104) by membrane or cell wall associated proteases (105);

FIG. 1c is a schematic depicting the mode of action of the antifungal conjugate (100) at the vicinity of the pathogen by proteases secreted by the pathogen (105);

FIG. 1d is a schematic depicting the mode of action of the antifungal conjugate (100) on host neutrophil ingested pathogen by host neutrophil specific proteases (105);

FIG. 2 provides the amino acid sequence of antibody fragment based antimicrobial conjugate represented by Seq. ID21;

FIG. 3a is a chromatogram of affinity purification by Ni-NTA (1st round) of conjugate Seq. ID 21 from solubilized inclusion bodies;

FIG. 3b is a representative SDS-PAGE image of affinity purified conjugate Seq. ID 21 from solubilized inclusion bodies;

FIG. 4a is a representative image of western blot of purified antibody fragment based antimicrobial conjugate of Seq. ID 21 (lane 1) and antibody fragment based antimicrobial conjugate of Seq. ID 21 exposed to P. aeruginosa where the peptide is released from the conjugate (lane 2);

FIG. 4b is a representative image of turbidity test of P. aeruginosa culture in the presence (Treated) and the absence (Control) of purified Seq. ID 21;

FIG. 5 is representative microbiological agar-plate assay to determine MIC-99 of purified Seq. ID 21, and Seq. ID 6;

FIG. 6a is a representative graph depicting the binding affinity of Seq. ID21 and Seq. ID 6 to whole cell Pseudomonas spp. in an ELISA assay;

FIG. 6b is a representative LC-MS/MS mass spectrogram identifying the target of Seq. ID6 camelid antibody as a C4 Decarboxylase ABC Transporter; and

FIG. 7 is a representative graph showing the kill kinetics of Seq. ID 21, Seq. ID 6, on P. aeruginosa with Histatin 5, positive control—Meropenem, and negative controls—media and culture.

### DETAILED DESCRIPTION OF THE INVENTION

The present invention now will be described hereinafter with reference to the detailed description, in which some, but not all embodiments of the invention are indicated. Indeed, the invention may be embodied in many different forms and should not be construed as limited to the embodiments set forth herein; rather, these embodiments are provided so that this disclosure will satisfy applicable legal requirements. The present invention is described fully herein with non-limiting embodiments and exemplary experimentation.

### Definitions

The term “antibody fragment” as used herein refers to polypeptides or proteins that bind to specific antigens. It also means immunoglobulins, not limited to polyclonal, monoclonal, chimeric, humanized antibodies, Fab fragments, F(ab′)2 fragments and likewise.

The term “antimicrobial peptide” as used herein refers to a polymer of amino acid residues typically ranging in length from 10 to about 50 which show antimicrobial properties by associating with membranes of microorganisms and causing membrane permeabilization, thereby killing the microorganisms.

The term “MIC” as used herein refers to minimal inhibitory concentration.

The term “MIC-99” as used herein refers to minimal inhibitory concentration for killing 99% microorganisms.

The term “MIC-50” as used herein refers to minimal inhibitory concentration for killing 50% microorganisms.

The term “next generation” as used herein refers to product that has been developed using latest technology to replace existing less efficient form of the drug.

The term “prodrug” as used herein refers to a compound that, after administration, is metabolized (i.e., converted within the body) into a pharmacologically active drug.

The term “in tandem” as used herein refers to one behind another. A sequence in tandem with another is adjacent sequences in continuation.

The term “VHH” as used herein refers to an antigen binding fragment of antibody which is composed only of heavy chains and does not comprise any light chains, it is also called as nanobody. Typically, about 30-40% of IgG antibody derived from camels comprises two heavy chains only. Each heavy chain comprises a variable region (encoded by VHH, D and J elements) and a constant region.

The term “virulent protease” as used herein refers to proteases naturally produced by pathogens to attack their host cells and aids in pathogenicity and subsequent colonization.

The company AbGenics Lifesciences Pvt. Ltd. has developed new generation of antibody fragment based antimicrobial conjugates known by the trademark AbTids® for providing a solution to management of drug-resistant Pseudomonas spp.

In the main embodiment of the invention, the invention provides a novel antibody fragment based antimicrobial conjugate selectively Pseudomonas spp., comprising of at least one antimicrobial peptide, at least one antibody fragment specific against the surface antigen of Pseudomonas spp., preferably, P. aeruginosa, and at least one signal protease cleavage sequence in tandem with at least one flexible polypeptide linker, with the signal protease cleavage sequence and the flexible polypeptide linker placed in between the antimicrobial peptide and antibody fragment.

The invention further relates to a novel antibody fragment based antimicrobial conjugate selective against Pseudomonas aeruginosa having amino acid sequence comprising of at least one antimicrobial peptide belonging to the group comprising of cationic histidine-rich antimicrobial peptides, mucin family of proteins, and human defensins, wherein, the cationic histidine-rich antimicrobial peptides are preferably Histatin family of peptides, more preferably, human Histatin-5 having amino acid sequence selected from the group consisting of Seq. ID. 1, and Seq. ID. 2 as listed in Table 1; the mucin family of proteins are Mucin 1-22, preferably human Mucin 7 having amino acid sequence of selected from the group consisting of Seq. ID. 3 and Seq. ID 4 as listed in Table 1; and the human defensins are preferably, human beta defensins, more preferably, human beta defensin having amino acid sequence represented by Seq. ID 5 listed in Table 1.

At least one antibody fragment, preferably a camelid VHH against Pseudomonas aeruginosa, wherein, the sequence of the VHH is selected from the group of sequence of amino acids represented by Seq. ID. 6, Seq. ID 7, Seq. ID. 8, Seq. ID 9, Seq. ID. 10, and Seq. ID 11 as listed in Table 1, preferably, Seq. ID. 6; at least one protease specific cleavage sequences susceptible to cleavage by proteases selected from the group consisting of membrane, cell wall associated, or secreted proteases of Pseudomonas spp., or host neutrophil proteases, wherein, the secreted virulent protease of Pseudomonas aeruginosa belongs to the group comprising of Protease IV, Alkaline Protease, Elastase A, and Elastase B, preferably, amino acid sequence represent by Seq. ID 12 and Seq. ID 13 susceptible to cleavage by virulent Elastase B, membrane or cell wall associated proteases of Pseudomonas spp. belongs to the group comprising of signal peptidase 3, preferably, amino acid sequence represent by Seq. ID 14 susceptible to cleavage by Pseudomonas spp. specific signal peptidase 3, and host neutrophil protease, preferably, amino acid sequence represented by Seq. ID 15 susceptible to cleavage by multiple proteases present in the neutrophil like Elastase, Proteinase 3, Matrix metalloproteinases 1 & 13, Thrombin, and Activated protein C, or a combination thereof; and at least one flexible polypeptide linker tandem to the protease cleavage sequence, wherein, the linker is selected from the group comprising of amino acid sequence with Glycine and Serine in tandem of formula {(G)₄S}ₙ, where n is 1-9, preferably Seq. ID 16, and Seq. ID 17, or from amino acid sequence represented by Seq. ID 18 where Glutamic acid can be substituted with Aspartate (D), or from Lysine rich sequences as represented by Seq. ID 19 or Seq. ID 20, or a combination thereof.

TABLE 1
List of amino acid sequences

| | |
|---|---|
| Sequence | |
| ID no. | Amino acid sequence |

Amino acid sequences of Antimicrobial peptide

| | |
|---|---|
| 1 | Met Xaa Asp Ser His Ala Arg His HisGly Tyr Lys |
| | Arg Lys Phe His Glu Lys His His Ser His Arg Gly Tyr |
| | XaaXaa, |
| | wherein “Xaa” is any naturally occurring amino acid |
| 2 | Met Gly Asp Ser His Ala Arg His HisGly Tyr Lys |
| | Arg Lys Phe His Glu Lys His Ser His Arg Gly Tyr |
| | Asp Val |
| 3 | XaaXaa Leu Ala His Gln Lys Pro Phe Ile Arg Lys Ser |
| | Tyr Lys Cys Leu His Lys Arg Cys Arg XaaXaa, |
| | wherein “Xaa” is any naturally occurring amino acid |
| 4 | GlyCys Leu Ala His Gln Lys Pro Phe Ile Arg Lys Ser |
| | Tyr Lys Cys Leu His Lys Arg Cys Arg |
| 5 | Gly Ile Gly Asp Pro Val ThrCys Leu Lys Ser Gly Ala |
| | Ile Cys His Pro Val PheCys Pro Arg Arg Tyr Lys Gln |
| | Ile GlyThrCysGly Leu Pro GlyThr Lys CysCys Lys Lys |
| | Pro |

Amino acid sequences of Camelid heavy chain antibody variable region fragment
(VHH) specific to Pseudomonas aeruginosa

| | |
|---|---|
| 6 | Asp Val Gln Leu Gln Glu Ser GlyGly Ala Ser Val Gln |
| | Pro GlyGly Ser Leu Leu Ile Ser Cys Glu Ala Ser Gly |
| | Leu Ala Ser Tyr Ser Asn Tyr Cys Ile Met TrpPhe Arg |
| | Gln Pro ProGly Lys Glu Arg Glu Gly Val Ala Gly Ile |
| | Asn Leu Arg Ser Gly Ile Thr Tyr Tyr Ala Glu Ala Val |
| | Arg Pro Arg PheThr Ile Ser Ala Asp Ser Val Asp Gly |
| | Arg Phe Ala Ile Ser Gln Asp Asn Ala Arg AsnThr Val |
| | Tyr Leu Gln Met Asn Ser Leu Lys Pro Glu Asp Thr Ala |
| | Ile Tyr TyrCys Ala AlaGlyAsn Leu CysGlyGly Ser Trp |
| | Ser Gly Tyr Arg Tyr TrpGly Gln GlyThr Gln Val Thr Val |
| | Ser Ser |
| 7 | Asp Val Gln Leu Gln Glu Ser GlyGlyGly Leu Val Gln Pro |
| | GlyGly Ser Leu Arg Leu Ser Cys Arg Ala Ser GlyTrpThr |
| | Ala Asp AsnTrp Tyr Met GlyTrpPhe Arg Gln Ser Pro Gly |
| | Lys Glu Arg Glu Ala Val Ala Ile IleGly His Arg Phe |
| | Asp ThrThr Tyr Tyr Ala Asp Ser Val Gln Gly Arg PheThr |
| | Ile Thr Gln Asp Asn Val Glu Lys Met Val Phe Leu Glu |
| | Met AsnAsn Leu Lys Pro Glu Asp Thr Ala Met Tyr TyrCys |
| | Ala Ile Gln Val Tyr AsnGlyGly Val Arg Pro Ser Pro Asp |
| | Ala Ala Lys Tyr Asn Tyr TrpGly Gln GlyThr Gln Val Thr |
| | Val Ser Ser |
| 8 | Asp Val Gln Leu Gln Glu Ser GlyGly Ala Ser Val Gln |
| | Val GlyGly Ser Leu Thr Leu Ser Cys Ser Thr Ser Lys |
| | Val Pro Asn Ile GlyCys Val ThrTrpPhe Arg Gln Gly Pro |
| | GlyGly Leu Gln Val Gly Ile Ala Ala Val Arg Thr Arg |
| | Tyr Gly Asp Thr Tyr Tyr Gln Asp Ser Ile Lys Gly Arg |
| | PheThr Ile Ser Arg Thr His ThrThr Glu Asn Leu Gln Met |
| | Asn Ala Leu Glu Pro Asp Asp Ala Ala Val Tyr Arg Cys |
| | Ala ThrThr Ser Lys Ser SerCys Tyr Ser GlyGly Ser |
| | TrpThr Leu Glu Asp Val Tyr Glu Tyr TrpGlyGln GlyThr |
| | Gln Val Thr Val Ser Ser |
| 9 | Asp Val Gln Leu Gln Asp Ser GlyGly Glu Ser Val Gln |
| | Ala GlyGly Ser Leu Arg Leu ThrCys Val Gly Ser GlyAsn |
| | Ser Phe Ile Arg Tyr Cys Met Ala TrpPhe Arg Gln Ala |
| | Pro Gly Lys Gln Arg Glu Glu Ile Val Glu Ser Gly Gln |
| | Phe Glu Phe Gln ThrTrpAsn Pro Asp Ser Val Lys Gly Arg |
| | PheThr Ile Ser Arg Asp Asn Ala Pro AsnThrGly Ser Leu |
| | His Met Asn Ser Leu Gln Ser Glu Asp Thr Ala Ala Tyr |
| | PheCys Ala AlaGly Met Ile Cys Pro Ile PheGly Arg Thr |
| | Gln Met Ser Ala Asp Met Asp Tyr TrpGly Arg GlyThr Gln |
| | Val Thr Val Ser Ser |
| 10 | Ser Val Gln Pro GlyGly Ser Leu Leu Ile Ser Cys Glu |
| | Ala Ser Gly Leu Ala Ser Tyr Ser Asn Tyr Cys Ile Met |
| | TrpPhe Arg Gln Pro ProGly Lys Glu Arg Glu Gly Val Ala |
| | Gly Ile Asn Leu Arg Ser Ser Ile Thr Tyr Tyr Ala Asp |
| | Ser Val Lys Ala Arg PheThr Ile Ser Ala Asp Ser Val |
| | Glu Gly Arg Phe Ala Ile Ser Gln Asp Lys Ser Arg |
| | AsnThr Val Leu Leu Gln Met Asn Ser Leu Lys Ala Glu |
| | Asp Thr Ala Asn Tyr TyrCys Ala AlaAla Val Cys Gln Ser |
| | Arg Tyr Met Ala His Asp Gln Val Ser Tyr Asn Tyr |
| | TrpGly Gln GlyThr Gln Val Thr Val Ser Ser |
| 11 | Asp Val Gln Leu Gln Glu Ser GlyGlyGly Ser Val Gln Ala |
| | GlyGly Ser Leu Arg Leu Ser Cys Val Val Ser Glu Tyr |
| | Arg Ala Cys Met GlyTrpPhe Arg Gln Ala Pro Gly Lys Glu |
| | Arg Glu Ala Val Ala Val Ile Gly Ser GlyThr Ser Thr |
| | Tyr Thr Ala Asp Ser Val Lys Gly Arg PheThr Ile Ser |
| | Arg Asp Asn Asp Arg Lys Thr Ala Thr Leu Gln Met Asp |
| | Ser Leu Glu Pro Glu Asp Thr Ala Ile Tyr TyrCys Ala |
| | Val Gly Arg AsnCys Lys Trp Pro Pro Leu AsnPheGly Ala |
| | ThrThrTrpGly Gln GlyThr Gln Val Thr Val Ser Ser |

Amino acid sequences of Protease cleavage sequence

| | |
|---|---|
| 12 | Arg GlyGlyGly Leu Ala |
| 13 | Ala GlyGly Leu Ala Pro |
| 14 | Ala Ser Ala Ala Leu Ala |
| 15 | Asn Ala Thr Leu Asp Pro Arg Ser Phe Leu Leu Arg Asn |

Amino acid sequences of flexible linker peptides

| | |
|---|---|
| 16 | GlyGlyGlyGly Ser |
| 17 | GlyGlyGlyGly Ser GlyGlyGlyGly Ser GlyGlyGlyGly Ser |
| 18 | Glu GluGly Glu Phe Ser Glu Ala Arg |
| | Where Glu is Glu (E) or Asp (D) |
| 19 | Gly Ser Ala Asp Asp Ala Lys Lys Asp Ala Ala Lys Lys |
| | Asp Gly Lys Ser |
| 20 | Ser Ser Ala Asp Asp Ala Lys Lys Asp Ala Ala Lys Lys |
| | Asp Asp Ala Lys Lys Asp Ala |

P. aeruginosa is a common nosocomial contaminant, and epidemics have been traced to many items in the hospital environment. Patients who are hospitalized for extended periods are frequently colonized by this organism and are at increased risk of developing infection. Eradication of Pseudomonas aeruginosa has become increasingly difficult due to its remarkable capacity to resist antibiotics. Strains of Pseudomonas aeruginosa are known to utilize their high levels of intrinsic and acquired resistance mechanisms to counter most antibiotics. These pathogens are now called Pan Drug Resistant and virtually uncontrollable.

Hence, one aspect of the present invention is to provide a novel molecule targeting P. aeruginosa in such a manner that the development drug-resistance in pathogens is not easily achievable and if drug-resistance is achieved it can be tackled by simple manipulations in the conjugate molecule to produce next generation drug molecules.

Peptide-based therapeutics to treat drug resistant pathogens might be an alternative to conventional antibiotics. Salivary innate immunity is the first line of defense against pathogens in the oral cavity. Histatin is normally present in the oral cavity. One of the most potent salivary peptides called Histatin 5 is a cationic histidine-rich peptide present in humans and higher primates and have both antibacterial and antifungal activity (Van et al., 1997, Biochem. J., 326: 39-45). The mode of action has been demonstrated to be by membrane disruption resulting in leakage from the cells and non-energy dependent lysis. Similarly, mucins are critical components of the gel layer that protect against invading pathogens. Different types of mucins exist throughout the body in various locations of which Mucin 7 is found in the oral cavity. However, such broad-spectrum peptide needs to be diligently inserted into an antimicrobial peptide conjugate to control its non-specific toxicity.

Hence, another aspect of the present invention is to design a novel antibody fragment based antimicrobial conjugate targeting P. aeruginosa in a highly specific manner which acts as a prodrug and is non-toxic to host. The prodrug is activated only upon interaction with pathogen to reduce toxicity to host cells. The conjugate comprising of at least one pathogen protease specific cleavage sequence, wherein, the protease cleavage specific sequence in tandem with a flexible polypeptide linker placed between the antimicrobial peptide and antibody fragment, which is cleaved upon interaction with membrane, cell wall associated, or secreted protease of Pseudomonas spp. The encounter of the conjugate with the Pseudomonas spp. due to antigen recognition by the antibody fragment of the conjugate initiates' cascade of reactions where upon the membrane or cell wall associated proteases or virulent secretory proteases cleave the protease specific cleavage sequence of the conjugate, thereby releasing the antimicrobial peptide from the antibody fragment. The antimicrobial peptide is now released from the prodrug and is capable to assert antimicrobial properties against the pathogen. Further, the protease cleavage sequence may be specific to host neutrophil proteases to clear pathogen which has been ingested by host neutrophils. Optionally, a combination comprising of conjugates having pathogen specific protease cleavage sequence, and conjugates having host neutrophil specific proteases cleavage sequence can be used to defend against both free pathogens and neutrophil ingested pathogens.

Different strains of P. aeruginosa secrete several extracellular proteolytic enzymes that have been implicated as virulence factors. Pseudomonas aeruginosa elastase B (also called LasB protease and pseudolysin) is one of the major proteins secreted into the environment which is a 33 kDa enzyme. Elastase B is involved in pathogenesis by degradation of human immunologically competent particles, cytokines, immunoglobulins, and others. Similarly, Pseudomonas specific signal peptidase is present on the outer membrane and transmembrane space that process the N terminal signal sequences of the secretory proteins before its eventual release into from the pathogen.

The antibody fragment based antimicrobial conjugates have application in urinary tract infections, lung infections in Cystic Fibrosis Transmembrane Conductance Regulator (CFTR) patients, epithelial infections in case of burns, diabetic and corneal ulcers, and other such infections caused by Pseudomonas aeruginosa.

The antibody fragment based antimicrobial conjugates are biological antimicrobial conjugates with a complex mode of action including immune engagement. Being a larger molecule with a size of ˜20 kDa, this does not penetrate the pathogen and instead acts from the outside lysing and neutralizing it. Furthermore, as it does not bind to a simple mutable target inside a cell, resistance against it will be difficult to develop and even if it does, the components of said conjugate can be shuffled or replaced or mutated rapidly to generate next generation of molecules within months as a response to the antibiotic resistance challenge. Said conjugates have been demonstrated to bind to and neutralize pathogens that are resistant to antibiotics and persisters that are difficult to be targeted by small molecule antibiotics.

### Example 1

### Antibody Fragment Based Antimicrobial Conjugate Design and its Mode of Action Against Pathogen

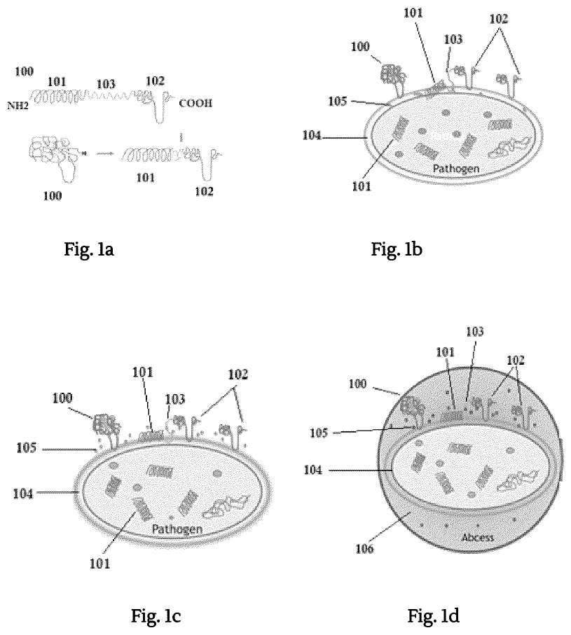

As depicted in FIG. 1a the antibody fragment based antimicrobial conjugate (100) comprises of antimicrobial peptide (101) followed by a linker (103) sequence and a pathogen specific antibody fragment (102). The linker (103) additionally comprises of a small protease cleavage sequence (103) susceptible to cleavage by pathogen specific proteases (105) such as membrane, cell wall associated, or secreted proteases, or host neutrophil specific proteases (105). The antibody fragment (102) is preferably a camelid VHH fragment targeting the pathogen surface or extracellular matrix antigen. The conjugate (100) is a prodrug which on encountering the pathogen initiates a cascade of reactions cleaving the protease cleavage sequence (103) which releases the antimicrobial peptide (101) from the antibody fragment (102).

The conjugate (100) may act against the pathogen in three different modes based on the kind of protease cleavage sequence (103).

Mode 1 is depicted in FIG. 1b, where the protease cleavage sequence (103) of the conjugate (100) is specific to membrane or cell wall associated proteases (105). The conjugate (100) targets the pathogen in the host organism by the anti-pathogen antibody fragment (102), which upon coming in contact with the pathogen membrane or cell wall (104) is susceptible to cleavage by membrane or cell wall associated proteases (105), thereby releasing the antimicrobial peptide (101) to act against the pathogen.

Mode 2 is depicted in FIG. 1c, where the protease cleavage sequence (103) of the conjugate (100) is specific to proteases secreted by pathogens (105). The conjugate (100) targets the pathogen in the host organism by the anti-pathogen antibody fragment (102), which upon coming in vicinity of the pathogen is susceptible to cleavage by pathogen secreted proteases, thereby releasing the antimicrobial peptide (101) to act against the pathogen.

Mode 3 is depicted in FIG. 1d, where the protease cleavage sequence (103) of the conjugate (100) is specific to host neutrophil specific proteases (105). The conjugate (100) is internalized by the host neutrophils (106) and inside the neutrophil the conjugate (100) targets neutrophil-ingested pathogen by the anti-pathogen antibody fragment (102). The conjugate after internalization by host neutrophil is susceptible to cleavage by host neutrophils proteases (105), thereby releasing the antimicrobial peptide (101) to act against the pathogen.

The antibody fragment based antimicrobial conjugate specific against Pseudomonas aeruginosa comprises of amino acids represented by Seq. ID 21,

Seq.ID 21:
Met Gly Asp Ser His Ala Lys Arg His HisGly Tyr Lys
Arg Lys Phe His Glu Lys His His Ser His Arg Gly
Tyr Asp Val Arg GlyGlyGly Leu Ala GlyGlyGlyGly
Ser GlyGlyGlyGly Ser GlyGlyGlyGly Ser His Met Asp
Val Gln Leu Gln Glu Ser GlyGly Ala Ser Val Gln Pro
GlyGly Ser Leu Leu Ile Ser Cys Glu Ala Ser Gly Leu
Ala Ser Tyr Ser Asn Tyr Cys Ile Met TrpPhe Arg Gln
Pro ProGly Lys Glu Arg Glu Gly Val Ala Gly Ile Asn
Leu Arg Ser Gly Ile Thr Tyr Tyr Ala Glu Ala Val
Arg Pro Arg PheThr Ile Ser Ala Asp Ser Val Asp Gly
Arg Phe Ala Ile Ser Gln Asp Asn Ala Arg AsnThr Val
Tyr Leu Gln Met Asn Ser Leu Lys Pro Glu Asp Thr
Ala Ile Tyr TyrCys Ala Ala GlyAsn Leu CysGlyGly
Ser Trp Ser Gly Tyr Arg Tyr TrpGly Gln GlyThr Gln
Val Thr Val Ser Ser Leu Glu

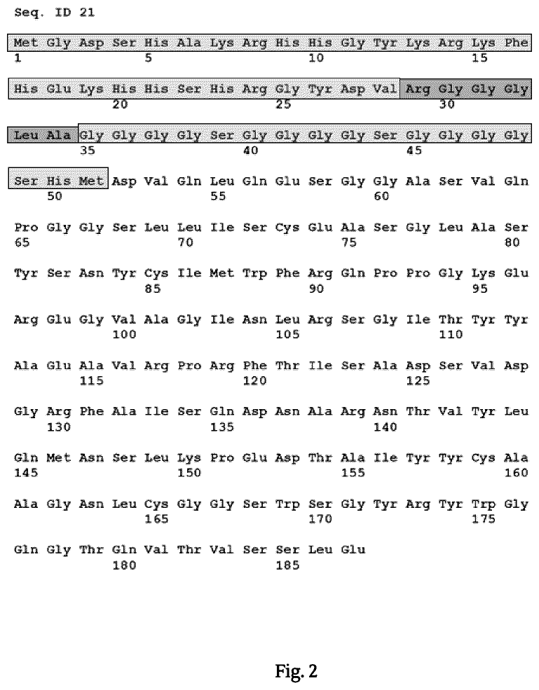

As depicted in FIG. 2, the Seq. ID21 comprises of 187 amino acids of which amino acids 1-28 correspond to the antimicrobial peptide, human Histatin 5, amino acids 29-34 (RGGGLA) is the Elastase B specific cleavage sequence, amino acid sequence 35-51 correspond to the linker with Glycine and Serine residues in tandem, and amino acid sequence 52-187 is Seq. ID6 which is a camelid heavy chain antibody variable region fragment (VHH) specific to Pseudomonas aeruginosa antigen C4 decarboxylase transporter responsible for nutrient uptake under anaerobic conditions.

The human Histatin 5 amino sequence is DSHAKRHHGYKRKFHEKHHSHRGY to with two amino acids MG at the N terminal and DV in the C terminal end have been added to give stability to the peptide after it has been released from the conjugate and also to facilitate fusion to the antibody during the cloning steps. This antimicrobial peptide can be placed on the N or the C terminal of the antibody connected by the same linker.

### Example 2

### Anti-Pseudomonas Camelid Heavy Chain Antibody Variable Region Fragment (VHH)

Heavy chain antibody based anti-Pseudomonas molecules was developed with the ability to kill the drug resistant Pseudomonas that possibly disrupt biofilms as well. For this purpose, camels were immunized with the extracts of Pseudomonas aeruginosa isolated from clinical samples. The antibody library was prepared in a phage display vector in E. coli and hits were isolated after panning against microbial cell wall components and strong binders assayed for their Pseudomonas neutralizing ability.

Camelid monoclonal antibodies are single heavy chain antibody molecules derived from camels, with low immune signature in humans, extremely small (14-17 kDa), with excellent stability and tissue penetrability properties. These antibodies do not need cold chain for transportation and remain stable for years at room temperature, a property, that can be exploited to develop and formulate stable antimicrobials. Furthermore, being small, they can be engineered to add value, have the ability of deep tissue penetration and disruption of biofilms. Six antibodies were isolated and sequenced with the Seq. ID6, Seq. ID 7, Seq. ID 8, Seq. ID 9, Seq. ID 10, and Seq. ID 11. These antimicrobial antibodies can be used to control topical as well as invasive Pseudomonas infections. The target for Seq. ID6 was identified to be a C4 decarboxylase transporter responsible for nutrient uptake under anaerobic conditions. This antibody was used as a backbone to produce the antibody fragment based antimicrobial conjugate.

### Example 3

### Expression and Purification Antibody Fragment Based Antimicrobial Conjugate of Seq. ID 21

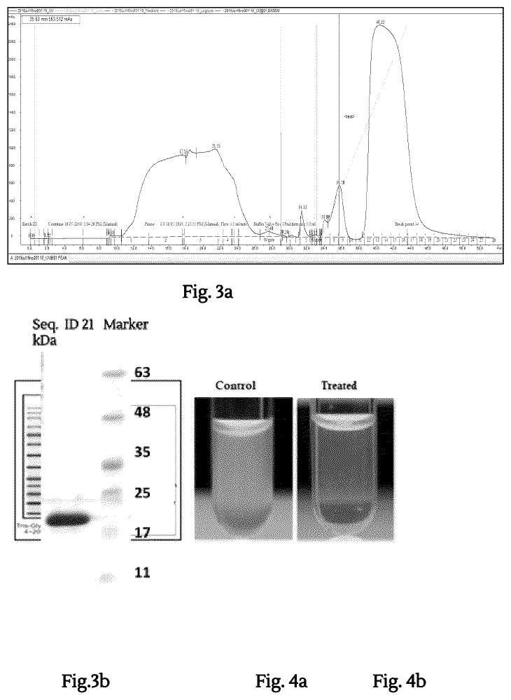

Conjugate with Seq. ID 21 (codes for a novel AbTid® targeting Pseudomonas aeruginosa) was expressed in pET28c+ vector in the E. coli BL21 (DE3) system as inclusion bodies, solubilized and purified using metal affinity and ion exchange chromatography and used for further analysis. The chromatogram of FIG. 3a shows affinity purification by Ni-NTA (1st round) of conjugate Seq. ID 21 from solubilized inclusion bodies through AKTA-prime plus along with SDS-PAGE showing purified Seq. ID 21 (˜20 kDa) as depicted in FIG. 3b.

### Example 4

### The Antibody Fragment Based Antimicrobial Conjugate is a Prodrug

The antibody fragment based antimicrobial conjugate which is initially a prodrug and inactive because the antimicrobial peptide is partially or wholly enclosed by the antibody component.

As depicted in FIG. 4a, the purified antibody fragment based antimicrobial conjugate of Seq. ID. 21 (Lane 1) was compared with antibody fragment based antimicrobial conjugate of Seq. ID. 21 exposed to P. aeruginosa (Lane 2) by western blot analysis using camelid antibody of Seq. ID 6. The purified Seq. ID 21 was larger in size compared to when exposed to P. aeruginosa which confirmed that the conjugate was cleaved when it encountered the proteases released by the pathogen.

FIG. 4b represents a turbidity test, wherein sample of the pathogen P. aeruginosa was either left untreated (Control) or was treated by addition of purified Seq. ID 21 (Treated) prodrug. The turbidity was visually reduced.

### Example 5

### A. Efficiency Test of the Antibody Fragment Based Antimicrobial Conjugate with Seq. ID 21

Microbiology assays were done with the purified VHH with Seq. ID 6 and the antibody fragment based antimicrobial conjugate of Seq. ID 21 to see their bactericidal activity. As depicted in FIG. 5, the MIC-99 of the Seq. ID6 antibody fragment alone was found to be 10 μM (anaerobic conditions) but the value was reduced by 20-fold for the conjugate of Seq. ID21 with a value less than 0.5 μM. The MIC-50 of Seq. ID 6 antibody fragment alone was less than 2.5 μM and that of Seq. ID 21 was less than 0.125 μM.

### B. Target Identification of the Conjugate with Seq. ID 21

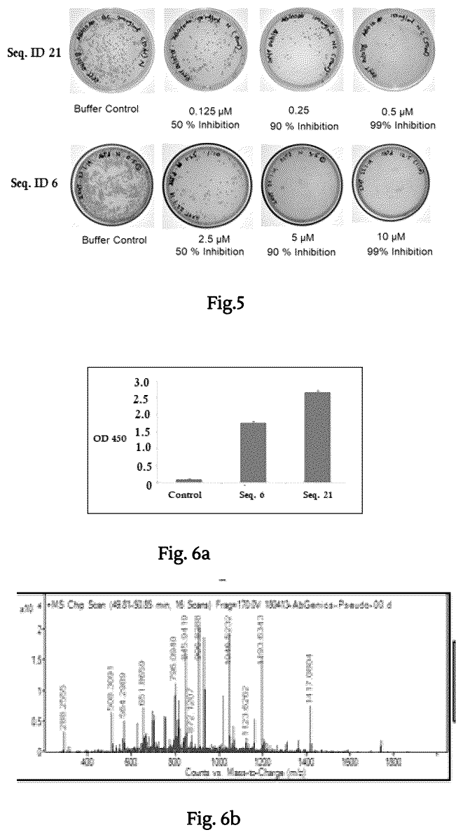

Whole cell ELISA using Pseudomonas spp. were used to determine the binding properties of the conjugate with Seq. ID 21, and VHH Seq. ID 6. As depicted in FIG. 6a, there was no loss of binding ability of the antibody when it was converted to conjugate with Seq. ID 21. Both the molecules Seq. ID 21 and Seq. ID6 showed the desired biological activity.

To identify the target of the Seq. ID 6 VHH, the cell wall lysate of Pseudomonas spp. was precipitated followed by LC-MS/MS and the mass spectrogram is shown in FIG. 6b. That identified target for Seq. ID 6 was a C4 Decarboxylase ABC Transporter present on the surface that is responsible for uptake of the C4 substrates: succinate fumarate and malate for anaerobic respiration. We characterized the target further by simple growth studies under anaerobic conditions using minimal media supplemented with these the C4 substrates as carbon source and by checking the growth inhibition after addition of the Seq. 6 VHH.

### C. Efficiency Test of the Antibody Fragment Based Antimicrobial Conjugate with Seq. ID 21

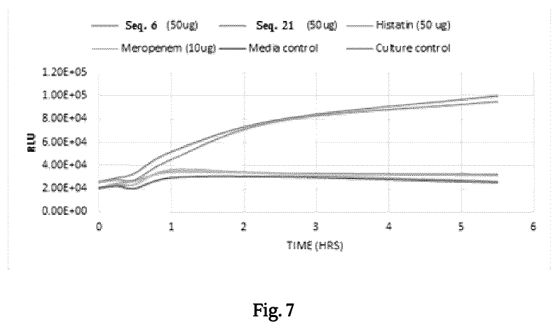

Kill kinetics was done by measuring the adenosine triphosphate (ATP) during different time points using the bacterial live dead assay. As shown in FIG. 7, the activity of the peptide Histatin 5 and the conjugate Seq. ID 21 was similar showing bactericidal activity was due to the released Histatin on contact with the Pseudomonas. A positive control used was antibacterial Meropenem drug to show the efficiency of killing. Distinct inhibition was seen at 1 hour after application of the compounds and there was no visible growth of the bacteria even after 5 hours. The VHH antibody Seq. ID 6 alone exerted a similar effect after a much longer interval due to gradual choking of the metabolic machinery due to interruption of the Krebs cycle.

While certain exemplary embodiments have been described and shown in the accompanying drawings, it is to be understood that such embodiments are merely illustrative of, and not restrictive on, the broad invention, and that this invention not be limited to the specific constructions and arrangements shown and described, since various other changes, combinations, omissions, modifications and substitutions, in addition to those set forth in the above paragraphs, are possible. Those skilled in the art will appreciate that various adaptations and modifications of the just described embodiments can be configured without departing from the scope and spirit of the invention. Therefore, it is to be understood that, within the scope of the appended claims, the invention may be practiced other than as specifically described herein.

We claim:

1. An antibody fragment based antimicrobial conjugate selectively targeting Pseudomonas spp. comprising of:
at least one antimicrobial peptide at one end of the conjugate;
at least one antibody fragment at the other end of the conjugate, preferably a camelid heavy chain antibody variable region fragment (VHH) specific against surface antigen of Pseudomonas spp.;
at least one signal protease cleavage sequence susceptible to cleavage by proteases selected from the group consisting of membrane proteases, cell wall associated proteases, and secreted proteases of Pseudomonas spp., and host neutrophil proteases; and
at least one flexible polypeptide linker in tandem with the signal protease cleavage sequence, with the signal protease cleavage sequence and the polypeptide linker placed in between the antimicrobial peptide and antibody fragment,
wherein,
said antimicrobial peptide belongs to the group comprising of cationic histidine-rich antimicrobial peptides, more particularly, human Histatin-5 represented by amino acids selected from the group comprising of Seq. ID 1 and Seq. ID 2;
said antibody fragment, preferably, VHH fragment targeting Pseudomonas aeruginosa is represented by Seq. ID 6 targeting C4 decarboxylase transporter;
said protease specific cleavage sequence is susceptible to cleavage by Pseudomonas aeruginosa virulent protease, Elastase B represented by Seq. ID 12;
the flexible polypeptide linker is amino acid sequence with Glycine and Serine in tandem of formula {(G)₄S}n, where n is 1-9, preferably Seq. ID 16, and Seq. ID 17;
the amino acid sequence of the conjugate is represented by Seq. ID 21;
said conjugate with Seq. ID 21 is specific against Pseudomonas spp., preferably, Pseudomonas aeruginosa, having in vitro MIC-99 against Pseudomonas aeruginosa of 0.5 μM, and MIC-50 less than 0.125μM; and
the in vitro MIC-99 of the VHH represented by Seq. ID 6 against Pseudomonas aeruginosa is 10 μM, and MIC-50 is less than 2.5 μM.

2. The antibody fragment based antimicrobial conjugate as claimed in claim 1, wherein, said antimicrobial peptide belongs to the group comprising of cationic histidine-rich antimicrobial peptides represented by amino acid sequence Seq. ID 1, and Seq. ID 2; mucin family of proteins represented by amino acid sequence Seq. ID 3, and Seq. ID 4, said human beta defensins, preferably, amino acid sequence Seq. ID 5.

3. The antibody fragment based antimicrobial conjugate as claimed in claim 1, wherein, the VHH fragment targeting Pseudomonas aeruginosa derived from immunized Camelus dromedarius having amino acid sequence represented by Seq. ID 6, Seq. ID 7, Seq. ID 8, Seq. ID 9, Seq. ID 10, and Seq. ID 11.

4. The antibody fragment based antimicrobial conjugate as claimed in claim 1, wherein, the protease specific cleavage sequence is susceptible to cleavage by proteases selected from the group consisting of proteases secreted by Pseudomonas aeruginosa belonging to the group comprising of Protease IV, Alkaline Protease, Elastase A, and Elastase B, preferably, amino acid sequence represent by Seq. ID 12 and Seq. ID 13 susceptible to cleavage by virulent Elastase B; or membrane or cell wall associated proteases of Pseudomonas spp. comprising of signal peptidase 3, preferably, amino acid sequence represented by Seq. ID 14 susceptible to cleavage by Pseudomonas spp. specific signal peptidase 3; or host neutrophil protease having amino acid sequence represented by Seq. ID 15; or a combination thereof.

5. The antibody fragment based antimicrobial conjugate as claimed in claim 1, wherein, the flexible polypeptide linker is selected from the group comprising of amino acid sequence with Glycine and Serine in tandem of formula {(G)4S}n, where n is 1-9, preferably Seq. ID 16, and Seq. ID 17, or from amino acid sequence represented by Seq. ID 18 where Glutamic acid can be substituted with Aspartate (D), or from Lysine rich sequences as represented by Seq. ID 19 or Seq. ID 20, or a combination thereof.

6. The antibody fragment based antimicrobial peptide and antibody conjugate as claimed in claim 1, wherein the conjugate is a non-toxic prodrug and gets activated only upon interaction of the VHH of the conjugate with Pseudomonas spp., thereby initiating a cascade of reactions leading to cleavage of the protease cleavage site of the conjugate releasing the antimicrobial peptide from the conjugate to act against the Pseudomonas spp.

7. The antibody fragment based antimicrobial conjugates claimed in claim 1, wherein, the antibody fragment is derived from a library of VHH fragments from camelids selected from the group comprising of dromedary camel, bactrian camels, wild or feral camels, llamas, alpacas, vicunas, or guanacos, preferably Camelus dromedarius.

8. The antibody fragment based antimicrobial conjugates claimed in claim 1, wherein, the conjugate can constitute pharmaceutical compositions for topical application, systemic delivery, or oral consumption.

9. The antibody fragment based antimicrobial conjugates claimed in claim 1, wherein, the conjugate can constitute formulations for coating medical implants to reduce infections.
